(19) United States
(12) Patent Application Publication
(10) Pub. No.: US 2026/0166064 A1
(43) Pub. Date: Jun. 18, 2026

(54) METHOD FOR PREPARING LIQUID INOSITOL COMPOSITION AND LIQUID INOSITOL COMPOSITION PREPARED THEREBY

(71) Applicant: INERTIA INC., Daejeon (KR)

(72) Inventors: Hyo Yi KIM, Daejeon (KR); Ji Hye PARK, Daejeon (KR); Eun Bie KO, Daejeon (KR); Seung Min LEE, Daejeon (KR)

(73) Assignee: INERTIA INC., Daejeon (KR)

(21) Appl. No.: 19/370,668

(22) Filed: Oct. 27, 2025

(30) Foreign Application Priority Data
Dec. 12, 2024 (KR) 10-2024-0184472

(51) Int. Cl. A61K 31/7004; A61K 9/08; A61K 47/36; A61K 47/42

(52) U.S. Cl. CPC A61K 31/7004; A61K 9/08; A61K 47/36; A61K 47/42

(57) ABSTRACT

The present invention relates to a method for preparing a liquid inositol composition and to the liquid inositol composition prepared thereby, wherein the liquid inositol composition includes 5 to 30 wt % of inositol; and one or more polysaccharides selected from the group consisting of an oligosaccharide, cyclodextrin, and allulose (D-psicose).

### CROSS-REFERENCE TO RELATED APPLICATION

This application claims priority to and the benefit of Korean Patent Application No. 10-2024-0184472, filed on Dec. 12, 2024, the disclosure of which is incorporated herein by reference in its entirety.

### BACKGROUND

### 1. Field of the Invention

The present invention relates to a method for preparing a liquid inositol composition and a liquid inositol composition prepared thereby, and more particularly, to a method for preparing a liquid inositol composition, which is stable because the composition does not generate crystallization or precipitates for a long period of time, and which is stable even when the composition is mixed with an active ingredient such as albumin, and a liquid inositol composition prepared thereby.

### 2. Discussion of Related Art

Inositol, which could conventionally be ingested through pharmaceuticals or health supplements, has only been available in solid forms such as powder, tablets, and granules. There have been no liquid inositol products available for oral consumption. Since inositol is a water-soluble compound, its absorption rate increases when consumed with a sufficient amount of water. Manufacturing it in a liquid form allows for convenient intake anywhere without the need to carry water and offers the advantage of rapid absorption without going through complex metabolic processes. In addition, when taken together with albumin, it can help alleviate gestational diabetes and be effective for women with insulin resistance. When combined with tartaric acid choline, folic acid, albumin, and vitamins B2, B6, and B12, folic acid becomes activated and can produce synergistic effects.

However, in conventional processes, high-temperature sterilization in accordance with the Food Code is essential. Under conditions above 70° C., inositol dissolves, but during the cooling process to room temperature, crystallization (precipitation) occurs, presenting a problem.

Furthermore, when albumin is mixed with an inositol solution for dissolution, coagulation or excessive foaming begins to occur after about 20 minutes of heating, posing another problem.

### RELATED ART DOCUMENTS

### Patent Document

Japanese Patent Publication No. 2019-033734

### SUMMARY OF THE INVENTION

Accordingly, the object of the present invention is to provide a method for preparing a liquid inositol composition that does not generate crystallization (precipitates) or foaming (bubbles) over an extended period, as well as to provide the liquid inositol composition produced thereby.

The objectives of the present invention are not limited to the technical problems mentioned above, and other technical tasks not explicitly stated herein will be clearly understood by those skilled in the art from the following description.

To solve the above problem, a method for preparing a liquid inositol composition according to an embodiment of the present invention includes: (a) heating a liquid composition including inositol and a polysaccharide; and (b) cooling the heated liquid composition.

Inositol may refer to one or more of the nine stereoisomers with a chemical formula of C₆H₁₂O₆, but specifically, it may refer to myo-inositol or cyclohexane-1,2,3,4,5,6-hexol (cis-1,2,3,5-trans-4,6-cyclohexanhexol).

Inositol may be included in an amount of 5 to 30 wt % based on the total liquid composition. Specifically, inositol may be included in an amount of 5 to 25 wt %, 5 to 20 wt %, 5 to 15 wt %, 5 to 10 wt %, 10 to 30 wt %, 10 to 25 wt %, 10 to 20 wt %, 10 to 15 wt %, 15 to 30 wt %, 15 to 25 wt %, 15 to 20 wt %, 20 to 30 wt %, 20 to 25 wt %, or 25 to 30 wt %.

In general, when inositol is dissolved in water in an amount of 5 wt % or more as shown in FIG. 1, heated and then cooled to room temperature, a supersaturation phenomenon is observed, and precipitates may be produced when inositol is present in an amount of 14 wt % or more. That is, the method according to the embodiments of the present invention may obtain a liquid composition in which 5 wt % or more of inositol is also completely dissolved without crystals or precipitates, and it will be obvious to a person skilled in the art that it is also possible to prepare a liquid composition containing 5 wt % or less of inositol. Therefore, inositol may be included in an amount of 30 wt % or less, 25 wt % or less, 20 wt % or less, 15 wt % or less, 10 wt % or less, or 5 wt % or less based on the total liquid composition.

The polysaccharide may include one or more selected from the group consisting of an oligosaccharide, cyclodextrin, allulose (D-psicose), and an orange concentrate.

The oligosaccharide may specifically be a (mixed) oligosaccharide including one or more selected from the group consisting of fructo-oligosaccharides (FOS), galactooligosaccharides (GOS), mannan oligosaccharides (MOS), xylooligosaccharides (XOS), and maltooligosaccharides.

The cyclodextrin may specifically include one or more of α-cyclodextrin, β-cyclodextrin, and γ-cyclodextrin.

As the orange concentrate, it is possible to use an orange concentration that satisfies one or more, two or more, three or more, four or more, or all of the following (1) to (5) conditions.
(1) Brix refractometer: 64 to 66 Brix
(2) Acidity citric: 3.5 to 5 a.c.a/100 g
(3) Ratio: 14 to 18 Brix/acidity
(4) pH: 4 or less
(5) Pulp: 4 to 12%

For the oligosaccharide, cyclodextrin, allulose, and orange concentrate, it is possible to use a raw material having a sugar content of 25 to 35 wt %, 70 to 80 wt %, 0 wt %, and 60 to 70 wt %, respectively, per raw material. In exemplary embodiments, it is possible to use a raw material of an oligosaccharide, cyclodextrin, allulose, or an orange concentrate having a sugar content of 31 wt %, 75.5 wt %, 0 wt %, and 65 wt %, respectively, per raw material, but the embodiments of the present invention are not limited thereto. The polysaccharide may be included in an amount of 1 to 20 wt % based on the total liquid composition. Specifically, the polysaccharide may be included in an amount of 1 to 15 wt %, 1 to 10 wt %, 1 to 5 wt %, 5 to 20 wt %, 5 to 15 wt %, 5 to 10 wt %, 10 to 20 wt %, or 10 to 15 wt %. More specifically, the polysaccharide may be included in an amount of 12 to 20 wt %, 12 to 17 wt %, 12 to 14 wt %, 14 to 20 wt %, 14 to 17 wt %, or 17 to 20 wt %.

In an embodiment, the liquid composition may include an oligosaccharide. Alternatively, the liquid composition may include an oligosaccharide and an orange concentrate. Alternatively, the liquid composition may include allulose and an orange concentrate. Alternatively, the liquid composition may include an oligosaccharide, cyclodextrin, allulose, and an orange concentrate.

The liquid composition may include 4 wt % or more, specifically 4 to 9 wt % of an oligosaccharide.

The liquid composition may include 5 wt % or more, specifically 5 to 9 wt % of cyclodextrin.

The liquid composition may include 3 wt % or more, specifically 3 to 9 wt % of allulose.

The liquid composition may include 5 wt % or more, specifically 5 to 9 wt %, and more specifically about 5 wt % of an orange concentrate.

In some embodiments, the liquid composition may include 4 wt % or more of an oligosaccharide, 5 wt % or more of cyclodextrin, 3 wt % or more of allulose, and 5 wt % or more of an orange concentrate, and the total sum of the polysaccharide content may be 12 to 20 wt %.

Alternatively, the liquid composition may include 5 wt % or more of an oligosaccharide, 5 wt % or more of cyclodextrin, 5 wt % or more of allulose, and 5 wt % or more of an orange concentrate, and the total sum of the polysaccharide content may be 12 to 20 wt %.

Alternatively, the liquid composition may include 7 wt % or more of an oligosaccharide and 5 wt % or more of an orange concentrate, and the total sum of the polysaccharide content may be 12 to 20 wt %.

Alternatively, the liquid composition may include 9 wt % or more of an oligosaccharide and 5 wt % or more of an orange concentrate, and the total sum of the polysaccharide content may be 12 to 20 wt %.

Alternatively, the liquid composition may include 9 wt % or more of allulose and 5 wt % or more of an orange concentrate, and the total sum of the polysaccharide content may be 12 to 20 wt %.

In exemplary embodiments, the liquid composition may include 4 to 5 wt % of an oligosaccharide, about 5 wt % of cyclodextrin, 3 to 5 wt % of allulose, and about 5 wt % of an orange concentrate.

Alternatively, the liquid composition may include 7 to 9 wt % of an oligosaccharide and about 5 wt % of an orange concentrate.

Alternatively, the liquid composition may include about 9 wt % of allulose and about 5 wt % of an orange concentrate.

In other embodiments of the present invention, a monosaccharide and/or a disaccharide such as glucose and fructose may be used together with or instead of a polysaccharide.

The liquid composition may be an aqueous solution in which inositol and a polysaccharide are at least partially dissolved or mixed in water (particularly purified water). However, the liquid composition is not limited thereto, and various solvents capable of adequately dissolving inositol and a polysaccharide may be used.

In some embodiments of the present invention, the liquid composition may further include one or more selected from the group consisting of a carob extract, choline, and folic acid together with a polysaccharide. The carob extract may be specifically chiro-inositol, more specifically D-chiro-inositol. The choline may specifically be choline bitartrate, but is not limited thereto.

Since the carob extract, choline, and folic acid are known to act synergistically with inositol in the human body, they may enhance the marketability of a liquid inositol composition when added, and they may also be provided as a stable liquid composition together with inositol by the method according to the embodiments of the present invention.

The carob extract may be added such that the weight ratio of carob extract to inositol is 1:10 to 1:100, 1:20 to 1:60, or 1:30 to 1:50, the choline may be added such that the weight ratio of choline to inositol is 1:5000 to 1:15000, 1:6000 to 1:12000, or 1:7000 to 1:9000, and the folic acid may be added such that the weight ratio of folic acid to inositol is 1:500 to 1:2000, 1:700 to 1:1500, or 1:900 to 1:1100.

The heating of the liquid composition of inositol is an essential step for high-temperature sterilization according to the Food Code, and the heating temperature may be 60 to 120° C., 60 to 110° C., 60 to 100° C., 60 to 90° C., 60 to 80° C., 60 to 70° C., 70 to 120° C., 70 to 110° C., 70 to 100° C., 70 to 90° C., 70 to 80° C., 80 to 120° C., 80 to 110° C., 80 to 100° C., 80 to 90° C., 90 to 120° C., 90 to 110° C., 90 to 100° C., 100 to 120° C., 100 to 110° C., or 110 to 120° C. Specifically, the heating may be performed by a method of heating in a water bath at the above temperature.

To preserve components, the heating time may be less than 60 minutes, less than 55 minutes, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, less than 5 minutes, less than 4 minutes, less than 3 minutes, less than 2 minutes, or less than 1 minute, and may be 5 seconds or more.

The cooling of the heated liquid composition may be cooling the heated liquid composition to any temperature less than the heating temperature or at room temperature. In this case, the heated liquid composition may be cooled while filled in a package such as a pouch or bottle of a final product, so that the liquid composition may be distributed as a final product immediately after cooling.

Inositol may produce crystals during the cooling process after heating for high-temperature sterilization, but a polysaccharide included with inositol adheres to the surface of a sugar in which crystallization occurs to inhibit crystallization, allowing inositol to be liquefied without precipitates.

A method for preparing a liquid inositol composition according to another embodiment of the present invention includes: (a) heating a liquid composition including inositol and a polysaccharide; (b) adding albumin to the heated liquid composition; (c) stirring the liquid composition to which the albumin is added; (e) degassing the stirred liquid composition under vacuum; and (d) cooling the degassed liquid composition.

Since steps (a) and (d) are the same as those described above, a detailed description thereof will be omitted.

The stirring speed may be 5 to 2,000 rpm, 5 to 1,000 rpm, 5 to 500 rpm, or 5 to 100 rpm, and specifically 5 to 50 rpm. When the stirring speed is within the above range, bubbles may be minimized, and when the stirring speed exceeds the above range, a large amount of bubbles are generated, so it may be difficult to prepare the liquid composition.

The stirring time may be 30 to 120 minutes. Specifically, the stirring time may be 30 to 100 minutes, 30 to 80 minutes, 30 to 60 minutes, 30 to 40 minutes, 50 to 120 minutes, 50 to 100 minutes, 50 to 80 minutes, 50 to 60 minutes, 70 to 120 minutes, 70 to 100 minutes, 70 to 80 minutes, 90 to 120 minutes, or 90 to 100 minutes.

In particular, albumin may generate bubbles very vigorously during stirring, which may be problematic during preparation and filling, and in a typically used propeller-type stirrer, excessive bubbles may be formed as external air is introduced due to the occurrence of a liquid level drop during stirring. Accordingly, in some embodiments of the present invention, it is possible to use a stirrer having a slow speed during stirring and capable of minimizing the liquid level drop, or a stirrer in which a container rotates together. In particular, a paddle-type impeller, specifically a triple paddle impeller as shown in FIG. 2, may be used to minimize bubbles, but the embodiments of the present invention are not limited thereto.

If albumin is added to the liquid composition when the temperature is too high, the albumin may be coagulated by substances having a thiol group in the composition, so the albumin may be added when the temperature is 90° C. or less, 80° C. or less, or 70° C. or less. Therefore, albumin may be added after allowing the liquid composition to stand until the temperature falls below the corresponding range or using a cooling apparatus, and the like.

The albumin may specifically be alpha-lactalbumin, but the embodiments of the present invention are not limited thereto.

Albumin may be added in an amount such that the weight ratio of albumin to inositol is 1:20 to 1:100.

Albumin, when acting together with inositol, is known to be useful in treating gestational diabetes and to enhance efficacy in women with inositol resistance, and thus may enhance the marketability of a liquid inositol composition, and albumin may be provided as a stable liquid composition together with inositol by the method according to the embodiments of the present invention.

In some embodiments of the present invention, one or more selected from the group consisting of vitamin A, vitamin B2, vitamin B6, vitamin B12, vitamin C, vitamin D, and folic acid may be further added together with the albumin. However, the embodiments of the present invention are not limited thereto.

Degassing is a step for minimizing bubbles in the liquid composition so that a stable liquid composition can be filled without any problem even when albumin is added, and may be performed under 0 MPa or less, −0.5 to 0 MPa, or −0.1 to 0.01 MPa.

The liquid inositol composition prepared by the method described above does not generate crystallization or precipitates, or does not coagulate or has no or reduced bubbles (foam) even when mixed with albumin. Specifically, the liquid inositol composition may not generate crystallization at −20 to 40° C. for 3 months or longer. The absence of crystallization may mean that no residue remains when the composition is filtered through a 20 mesh filter.

A health functional food composition containing inositol according to an embodiment of the present invention may include, as an active ingredient, the liquid inositol composition prepared by the aforementioned method.

The health functional food composition can be manufactured and administered in various oral and non-oral dosage forms. When formulated, it may be prepared using diluents or excipients commonly employed, such as fillers, extenders, binders, wetting agents, disintegrants, and surfactants. Solid formulations for oral administration include tablets, pills, powders, granules, and capsules, which are prepared by mixing one or more compounds with at least one excipient. In addition to simple excipients, lubricants may also be used. Liquid formulations for oral administration include suspensions, solutions, emulsions, and syrups, which may contain commonly used diluents and excipients.

Alternatively, the composition may contain various additional ingredients such as flavoring agents or natural carbohydrates, similar to ordinary beverages. Furthermore, it may include additional components such as nutrients, vitamins, flavoring agents, colorants, thickening agents, viscosity enhancers, pH regulators, stabilizers, preservatives, and carbonation agents.

Specific details of other embodiments are included in the detailed description below.

### BRIEF DESCRIPTION OF THE DRAWINGS

FIG. 1 is a photograph showing the results obtained by dissolving inositol in water according to its concentration, followed by heating and cooling (left), and the results of preparing a liquid composition by heating and cooling inositol at 20 wt % (right).

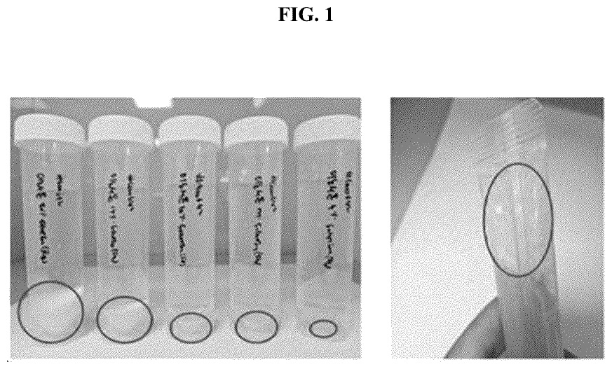

FIG. 2 illustrates the structure of a paddle-type impeller (triple paddle impeller) that can be used according to one embodiment of the present invention.

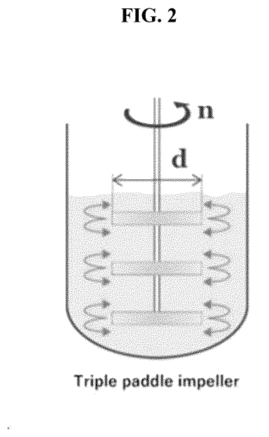

### DETAILED DESCRIPTION OF EXEMPLARY EMBODIMENTS

The advantages and features of the present invention, and methods for achieving them, will become apparent with reference to the embodiments described in detail below. However, the present invention is not limited to the embodiments disclosed herein and may be implemented in various other forms. The embodiments are merely provided to ensure a complete disclosure of the invention and to fully convey the scope of the invention to those skilled in the art, and the invention is defined only by the scope of the claims.

The terminology used in this specification is intended to describe particular embodiments and is not meant to limit the invention. As used herein, the term “and/or” includes each and all combinations of the items mentioned. Unless otherwise specified, the singular forms also include the plural forms. The terms “comprises” and/or “comprising” as used herein do not exclude the presence or addition of one or more other components beyond those mentioned.

A numerical range indicated by “-” or “to” includes the lower and upper limits unless otherwise stated. The terms “about” or “approximately” refer to a value or range within 20% of the stated value or range.

In this specification, when a range is described for a variable, it should be understood to include all values within the endpoints of the stated range. For example, the range “5 to 10” includes the values 5, 6, 7, 8, 9, and 10, as well as any subranges such as 6 to 10, 7 to 10, 6 to 9, or 7 to 9, and any intermediate values such as 5.5, 6.5, 7.5, 5.5 to 8.5, or 6.5 to 9. Similarly, the range “10% to 30%” includes all integer values from 10% to 30% (e.g., 11%, 12%, 13%, etc.), as well as any subranges (e.g., 10% to 15%, 12% to 18%, 20% to 30%) and intermediate values (e.g., 10.5%, 15.5%, 25.5%) within the stated range.

In describing the components of an embodiment of the present invention, terms such as first, second, A, B, (a), (b), etc. may be used merely to distinguish one element from another. These terms do not imply any specific nature, sequence, or order of the components.

Unless otherwise defined, all terms (including technical and scientific terms) used herein have meanings commonly understood by those skilled in the art to which the invention pertains. Terms defined in generally used dictionaries should be interpreted in a manner consistent with their ordinary meanings and not in an idealized or overly broad sense unless explicitly defined otherwise.

In describing the embodiments of the present invention, detailed descriptions of well-known structures or functions may be omitted if they would unnecessarily obscure the understanding of the invention.

As used herein, the term “prevention” refers to suppressing or delaying the onset of symptoms or diseases in an individual who does not yet exhibit such symptoms or diseases but is at risk of developing them.

The terms “treatment” or “improvement” refer to any act that alleviates or beneficially alters a condition in an individual, including (a) suppression of the progression or aggravation of symptoms or disease, (b) mitigation of symptoms or disease, or (c) elimination of symptoms or disease.

The term “level” may refer to the measurable amount or concentration of a specific factor as determined by quantitative analysis.

The term “isomer” refers to a compound or its salt having the same chemical or molecular formula but differing structurally or stereochemically. Such isomers include structural isomers such as tautomers, stereoisomers having asymmetric carbon centers such as R or S isomers, geometric isomers (trans, cis), and optical isomers (enantiomers).

Hereinafter, embodiments of the present invention will be described in detail with reference to manufacturing examples and experimental examples; however, it will be apparent that the effects of the invention are not limited to these examples.

### PREPARATION EXAMPLES

### Example 1

An aqueous solution was prepared by adding inositol to purified water to achieve a concentration of 15 wt %, followed by the addition of 5 wt % oligosaccharide. The mixture was heated in a water bath at 80° C., stirred at 30 rpm, and then cooled to room temperature. Myo-inositol was used as the inositol source.

### Example 2

The composition was prepared in the same manner as in Example 1, except that 5 wt % of cyclodextrin was added instead of oligosaccharide.

### Example 3

The composition was prepared in the same manner as in Example 1, except that 5 wt % of allulose was added instead of oligosaccharide.

### Example 4

An aqueous solution was prepared by adding inositol to purified water to achieve a concentration of 15 wt %, followed by the addition of 5 wt % oligosaccharide. The mixture was heated in a water bath at 80° C. and stirred at 30 rpm using a paddle-type impeller. Then, α-lactalbumin was added at a weight ratio of 1:50 (=α-lactalbumin: inositol). After degassing under a vacuum of −0.09 MPa, the mixture was cooled to room temperature.

### Comparative Example 1

The composition was prepared in the same manner as in Example 1, except that no oligosaccharide was added.

### Comparative Example 2

The composition was prepared in the same manner as in Example 4, except that stirring was performed at 60 rpm.

### Comparative Example 3

The composition was prepared in the same manner as in Example 4, except that a propeller-type impeller was used for stirring.

### Comparative Example 4

The composition was prepared in the same manner as in Example 4, except that the vacuum degassing step was omitted.

### Experimental Example 1: Evaluation of Precipitation and Foaming of the Liquid Inositol Compositions

The results of observing the compositions of inositol prepared in the above examples are shown in Table 1 below.

TABLE 1

| Classification | Precipitation | Foaming |
|---|---|---|
| Example 1 | Not observed | Almost none |
| Example 2 | Not observed | Almost none |
| Example 3 | Not observed | Almost none |
| Example 4 | Not observed | Almost none |
| Comparative | Large amount observed | Almost none |
| Example 1 | | |
| Comparative | Not observed | Small amount observed |
| Example 2 | | |
| Comparative | Small amount observed | Large amount observed |
| Example 3 | | |
| Comparative | Small amount observed | Large amount observed |
| Example 4 | | |

### Experimental Example 2: Evaluation of Crystallization According to the Type and Content of Polysaccharides

After preparing liquid inositol compositions using various types and contents of polysaccharides as shown in Table 2 below, the presence or absence of crystallization was observed. As a result, it was confirmed that no crystallization occurred for any of the various polysaccharide types and contents, as shown in Table 2.

TABLE 2

| | Oligo- | | | Orange | Presence |
|---|---|---|---|---|---|
| | saccharide | Cyclodextrin | Allulose | Concentrate | of |
| Example | (wt %) | (wt %) | (wt %) | (wt %) | Crystals |
| 5-1 | 4 | 5 | 3 | 5 | X |
| 5-2 | 5 | 5 | 5 | 5 | X |
| 5-3 | 7 | 0 | 0 | 5 | X |
| 5-4 | 9 | 0 | 0 | 5 | X |
| 5-5 | 0 | 0 | 9 | 5 | X |
| 5-6 | 0 | 0 | 0 | 0 | ◯ |
| 5-7 | 0 | 0 | 0 | 0 | ◯ |

Although the present invention has been described with reference to the above embodiments, these are merely illustrative examples and are not intended to limit the invention. It will be apparent to those skilled in the art that various modifications and applications not exemplified herein can be made without departing from the essential characteristics of the embodiments of the present invention. For example, each component specifically illustrated in the embodiments of the invention may be modified in practice, and such modifications and applications should be interpreted as falling within the scope of the invention as defined by the appended claims.

According to the embodiments of the present invention, it is possible to prepare a liquid inositol composition that does not generate crystallization (precipitates) or foaming (bubbles) even after heating and cooling for high-temperature sterilization.

The effects of the embodiments of the present invention are not limited to the examples described above, and various additional effects are included within the scope of this specification.

What is claimed is:

1. A method for preparing a liquid inositol composition, the method comprising: (a) heating a liquid composition comprising inositol and a polysaccharide; and
(b) cooling the heated liquid composition.

2. The method of claim 1, further comprising, between steps (a) and (b),
stirring the heated liquid composition;
adding albumin to the stirred liquid composition; and
degassing the liquid composition to which albumin is added under vacuum.

3. The method of claim 1, wherein the polysaccharide comprises one or more selected from the group consisting of an oligosaccharide, cyclodextrin, allulose (D-psicose), and an orange concentrate.

4. The method of claim 2, wherein the polysaccharide comprises one or more selected from the group consisting of an oligosaccharide, cyclodextrin, allulose (D-psicose), and an orange concentrate.

5. The method of claim 1, wherein inositol is included in an amount of 5 to 30 wt % in the liquid composition.

6. The method of claim 2, wherein inositol is included in an amount of 5 to 30 wt % in the liquid composition.

7. The method of claim 2, wherein the stirring speed is 5 to 50 rpm.

8. The method of claim 2, wherein the stirring is performed using a paddle-type impeller.

9. The method of claim 2, wherein a weight ratio of albumin to inositol is 1:20 to 1:100.

10. The method of claim 2, wherein the adding of the albumin is performed when a temperature of the liquid composition is 70° C. or less.

11. A liquid inositol composition comprising:
5 to 30 wt % of inositol; and
one or more polysaccharides selected from the group consisting of an oligosaccharide, cyclodextrin, allulose (D-psicose), and an orange concentrate,
wherein the liquid inositol composition does not generate crystallization at −20 to 40° C. for 3 months or longer.

12. The liquid inositol composition of claim 11, further comprising albumin, wherein a weight ratio of albumin to inositol is 1:20 to 1:100.
